(19) United States
(12) Patent Application Publication
(10) Pub. No.: US 2005/0137703 A1
(43) Pub. Date: Jun. 23, 2005

(54) Accommodative intraocular lens

(75) Inventor: Jin Hui Chen, Nashville, TN (US)

Correspondence Address: MORRIS MANNING & MARTIN LLP, 1600 ATLANTA FINANCIAL CENTER, 3343 PEACHTREE ROAD, NE, ATLANTA, GA, 30326-1044, US

(73) Assignee: Vanderbilt University, Nashville, TN (US)

(21) Appl. No.: 11/004,601

(22) Filed: Dec. 3, 2004

Related U.S. Application Data
(63) Continuation-in-part of application No. 10/474,988, filed on Apr. 9, 2004.
(60) Provisional application No. 60/527,399, filed on Dec. 5, 2003.

(51) Int. Cl. A61F 2/16

(52) U.S. Cl. 623/6.13; 623/6.37; 623/6.4

(57) ABSTRACT

An accommodative intraocular lens for implantation in an eye of a living subject having a lens capsule and a lens substance contained in the lens capsule. In one embodiment, the accommodative intraocular lens includes a lens structure having a geometry and a focal power associated with the geometry. The lens geometry is changeable in response to a force applied to the lens structure. The accommodative intraocular lens further includes a frame for engaging the lens structure with the lens capsule of the eye of the living subject such that contraction of the lens capsule pushes the lens structure contracting inwardly and relaxation of the lens capsule pulls the lens structure extending outwardly so as to change the geometry of the lens structure to adjust the focal power of the lens structure accordingly.

### CROSS-REFERENCE TO RELATED PATENT APPLICATION

This application is a continuation-in-part of U.S. patent application Ser. No. 10/474,988, filed Oct. 16, 2003, entitled “INTRAOCULAR LENS SYSTEM,” by Jin Hui Shen, the disclosure of which is hereby incorporated herein by reference in its entirety, which status is pending and itself claims the benefit, pursuant to 35 U.S.C. §119(e), of provisional U.S. patent application Ser. No. 60/284,359, filed Apr. 17, 2001, entitled “INTRAOCULAR LENS SYSTEM,” by Jin Hui Shen, which is incorporated herein by reference in its entirety. This application also claims the benefit, pursuant to 35 U.S.C. § 119(e), of provisional U.S. patent application Ser. No. 60/527,399, filed Dec. 5, 2003, entitled “ACCOMMODATIVE INTRAOCULAR LENS,” by Jin Hui Shen, which is incorporated herein by reference in its entirety.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, “[n]” represents the nth reference cited in the reference list. For example, [22] represents the 22nd reference cited in the reference list, namely, Shen J H, O'Day D M: Designing of an Accommodative Intraocular Lens. Invest Ophthalmol Vis Sci 43(Suppl):402. 2002.

### FIELD OF THE INVENTION

The present invention generally relates to an intraocular lens, and in particular to an accommodative intraocular lens.

### BACKGROUND OF THE INVENTION

Accommodation, or a change in the focus of the human lens, is a consequence of the ability of the lens to change its shape by contracting the capsule. This contraction function is what normally changes the shape of lens capsule in response to the need to accommodate.

The crystalline lens is one of the main optical elements in human vision. It provides the focus adjustment function in the eye. As shown in FIGS. 1A and 1B from reference [1], the lens 100 has a capsule 102 and lens substance 104. The lens 100 is suspended by zonules 106 from the ciliary processes 108. Normally, when the lens 100 is at a non-accommodating condition as shown in FIG. 1A, which means the eye is focused at a distance, the ciliary muscle 108 is at a relaxed condition. The shape of the lens 100 is relatively flat, which is determined by its own natural elasticity, and the lens 100 now has a lower focal power. When the eye looks at objects a short distance away as shown in FIG. 1B, however, the ciliary muscle 108 contracts, and the lens 100 tends to accommodate. For this to happen, the lens 100 has to increase its thickness. Correspondingly, there are a decrease in the diameter of the lens 100 and a decrease in the anterior and posterior surface radii, which are determined by the natural shape of capsule 102. As shown in FIG. 1B, in the act of accommodation, the anterior surface of the lens 100 becomes more convex axially, and the posterior surface of the lens 100 also becomes more convex. Consequently, a higher focal power for the lens 100 is created. The parameter changes during lens accommodation are listed in Table 1 [2].

TABLE 1
Lens parameter changes with accommodation.

| | | Unaccommodated | | Accommodated | | |
|---|---|---|---|---|---|---|
| | | condition | | condition | | Reference |
| Refracting power | | +19.11 | D | +33.06 | D | [1] |
| Focal length | | 43.707 | mm | 33.785 | mm | [2] |
| | | 69.908 | mm | 40.416 | mm | |
| Radius of lens surface | | 11.62 | mm | 6.90 | mm | [3] |
| | | 12 | mm | 5.0 | mm | |
| Thickness of the lens | | 3.66 | mm | 4.24 | mm | [3] |
| | | 3.84 | mm | 4.20 | mm | |
| Lens equatorial | 15 yr. | 9 | mm | 8 | mm | [4] |
| diameter | 43 yr. | 10.4 | mm | 9.4 | mm | |
| (lens from | 63 yr. | 10.8 | mm | 9.8 | mm | |
| different age) | | | | | | |

As people age, the amplitude of accommodation is gradually reduced due to changes in the lenticular factors such as a decrease in the elasticity modulus of the capsule, an increase in the elasticity modulus of the lens substance, a flattening of the lens, or a combination of them. FIG. 2, by Fincham [3], shows presbyopic changes in the amplitude of accommodation due to changes with age in the lens.

When a person ages, the substance of the person's natural lens gradually hardens, and may lose its accommodation function. Additionally, the person's vision is also reduced by cataract formation. Cataract surgery is then necessary to restore vision.

In modern cataract surgery, the cataractous substance of the lens is removed through an opening in the lens capsule. The now empty capsule of the lens is retained. The surgeon then replaces the lens contents with an artificial lens, which is positioned in the empty capsule. A typical procedure for a cataract surgery includes providing an opening at limbus, removal of the front portion of the lens capsule, ultrasonic fragmentation of the hard lens substance (nucleus), and implantation of an artificial intraocular lens.

Intraocular lenses (hereinafter “IOL”) are high optical quality lenses made of synthetic material such as Polymethylmethacrylate (Acrylic) (hereinafter “PMMA”), silicone, hydrogel or the like. The diameter of an IOL is normally 5 to 7 mm, and the lens dioptric power is matched to the need of the patient. Each IOL has two spring-like haptics, or loops, attached to the optic. When the IOL is inserted inside the lens capsule, the haptics help to position the optic lens in the center. Haptics material are PMMA, polypropylene, or polyamide. There are varieties of haptics designs among different IOLs. Some of the configurations are shown in FIG. 3. For examples, IOL 301 has optic 302 and haptics 304, where haptics 304 are J-shaped loops. Moreover, IOL 311 has haptics that are C-shaped loops, IOL 321 has haptics that are lone J-shaped loops, and IOL 331 has haptics that are closed loops.

Visual function following cataract and IOL implant surgery generally is good. However, among other things, a major disadvantage is the loss of accommodative capability that a natural lens can offer because the artificial intraocular lens has a fixed focusing power.

Previous research by R. F. Fisher [4] has showed that after extraction of the cataractous lens contents, the lens capsule still retains a certain level of the accommodative capability.

Efforts have been made to restore accommodation after cataract and implant surgery, which can be divided into the following categories:

1) Refill the lens with a synthetic material. This technique was first introduced by Kessler [7]. Efforts have been continued to improve the technology around the world, for examples, by a research group at Bascom Palmer Eye Institute, University of Miami, Fla. [8], and a research group in Japan [9, 10]. The normal procedure for this technique includes the steps of removing the crystalline lens through a small anterior capsular hole, and refilling the capsular bag with either precured silicone gel, or an inflatable endocapsular balloon. All of these studies showed that the refilled lens recovered accommodation to some extent, but the amount was not sufficient to be clinically useful. 2) Bifocal or multifocal intraocular lens. Bifocal or multifocal IOLs were first introduced clinically in 1987 by Keates et al. [11] . Currently, several different types of multifocal IOL have been developed, including the multizone bifocal lens [12, 13], the aspherical multifocal IOL [14], and the diffractive multifocal IOL [15-18]. Nevetheless, these IOLs can only give a patient two focus points and/or a limited focus range, and at each focus point, the patient can only get half of the incoming light energy. Consequently, at each focus distance, the images the patient has are blurry.

3) Accommodative intraocular lens. Several groups have been working along this line of research. For examples, one is in Japan [6, 19], and the other in the Netherlands [20]. In both studies, a movable optical lens is utilized in the direction of the axis of the eye, which is controlled by the ciliary muscle. While there was a limited amount of accommodative function shown, again no full accommodation was restored.

Recently, an accommodative IOL was proposed by Oliver Findl, M.D., of Vienna, Austria and published in Eye World in July 2000 [21]. As shown in FIG. 4, in Dr. Findl's IOL design, a fixed focus lens 402 is held by two pieces 404, 406 of ridged plastic holder, and the connection 408 between each plastic holder 404 or 406 and the lens 402 is flexible. When the ciliary muscle contracted, the IOL 400 will move forward. By this design, up to 2.5 D of the accommodation has been achieved. Still, no full scale of accommodation is available.

Shen and O'Day have designed an accommodative IOL [22]. It consists of six or eight eccentrically overlapped Gaussian lenses that are fixed on an elastic zigzag thin wire frame. The dimension of each Gaussian lens is about 6 mm in diameter and 100 μm in thickness. When ciliary muscle and the lens capsule contracts, it pushes the Gaussian lenses move toward concentric direction, thus create accommodation effect. In vitro test of this IOL in a simulated ocular environment has demonstrated that 0.8 mm change of the outer diameter could induce 1.1 mm focus distance change at the simulated retina position. However, the design of this IOL seems complicated.

Therefore, a heretofore unaddressed need still exists in the art to address the aforementioned deficiencies and inadequacies.

### SUMMARY OF THE INVENTION

In one aspect, the present invention relates to an accommodative intraocular lens for implantation in an eye of a living subject having a lens capsule and a lens substance contained in the lens capsule. In one embodiment, the accommodative intraocular lens includes a lens structure having a center of geometry, an inner surface defining a volume, an outer surface, a thickness defined therebetween the inner surface and the outer surface, and an edge, and a frame having a center of geometry, a plurality of inner ends and a plurality of outer ends. The plurality of inner ends of the frame are attached to the edge of the lens structure at a plurality of positions, respectively, such that the center of geometry of the frame overlaps substantially with the center of geometry of the lens structure. The plurality of outer ends of the frame are attached to an equator portion of the lens capsule at a plurality of positions, respectively. The volume of the lens structure is filled with an optically transparent liquid. The optically transparent liquid, in one embodiment, has a liquid gel.

The lens structure and the frame are adapted such that the lens structure has a contraction force directing inwardly to the center of geometry of the lens structure and the frame has an expansion force directing outwardly from the center of geometry of the frame, and when the lens capsule relaxes, the frame pulls the lens structure to be in a first state with an effective focal power, and when the lens capsule contracts and presses the fame inwardly to the center of geometry of the frame, the motion of the frame causes the lens structure to move inwardly to the center of geometry of the lens structure from the first state to a second state with an effective focal power that is different from the effective power of the lens structure at the first state. In one embodiment, the effective power of the lens structure at the second state is greater than the effective power of the lens structure at the first state.

The lens structure of the accommodative intraocular lens, in one embodiment, is convex. The edge of the lens structure is substantially circular. Each of the inner surface and the outer surface of the lens structure has a variable curvature and a projected geometric configuration of a circle. In one embodiment, the thickness of the lens structure is uniform. In another embodiment, the thickness of the lens structure is non-uniform. In one embodiment, the lens structure is made of an elastic silicone rubber. The elastic silicone rubber includes one of an elastomeric polydimethylsiloxane and a hydrogel.

The frame of the accommodative intraocular lens includes a structure that is symmetrical to the center of geometry of the frame. In one embodiment, the frame has a closed-loop structure. The closed-loop frame includes an elastic thin wire ring in a shape adapted for fitting to the equator portion of the lens capsule. In another embodiment, the frame has an open-loop structure.

In another aspect, the present invention relates to an accommodative intraocular lens for implantation in an eye of a living subject having a lens capsule and a lens substance contained in the lens capsule. In one embodiment, the accommodative intraocular lens includes a lens structure. The lens structure has a center of geometry, an inner surface defining a volume, an outer surface, a thickness defined therebetween the inner surface and the outer surface, and an edge. In one embodiment, the lens structure is convex. Each of the inner surface and the outer surface of the lens structure has a variable curvature and a projected geometric configuration of a circle. The thickness of the lens structure is either uniform or variable. The edge of the lens structure is substantially circular. In one embodiment, the lens structure is made of an elastic silicone rubber. The elastic silicone rubber includes one of an elastomeric polydimethylsiloxane and a hydrogel.

The accommodative intraocular lens further includes a ball lens. The ball lens has a center of geometry and a predetermined diameter, r, and is positioned in the volume of the lens structure with its center of geometry substantially overlapping with the center of geometry of the lens structure, where the rest of the volume of the lens structure is filled with a first gel. The ball lens includes a solid lens. In one embodiment, the ball lens is formed with a second gel that is harder than the first gel, where the first gel comprises an optically transparent liquid gel.

Additionally, the accommodative intraocular lens includes a frame having a center of geometry, a plurality of inner ends and a plurality of outer ends, where the plurality of inner ends of the frame are attached to the edge of the lens structure at a plurality of positions, respectively, such that the center of geometry of the frame overlaps substantially with the center of geometry of the lens structure, and the plurality of outer ends of the frame are attached to an equator portion of the lens capsule at a plurality of positions, respectively. The frame includes a structure that is symmetrical to the center of geometry of the frame. In one embodiment, the frame has a closed-loop structure. The closed-loop frame includes an elastic thin wire ring in a shape adapted for fitting to the equator portion of the lens capsule. In another embodiment, the frame has an open-loop structure.

In one embodiment, the lens structure and the frame are adapted such that the lens structure has a contraction force directing inwardly to the center of geometry of the lens structure and the frame has an expansion force directing outwardly from the center of geometry of the frame, and when the lens capsule relaxes, the frame pulls the lens structure to be in a first state with an effective focal power, and when the lens capsule contracts and presses the fame inwardly to the center of geometry of the frame, the motion of the frame causes the lens structure to move inwardly to the center of geometry of the lens structure from the first state to a second state with an effective focal power that is different from the effective power of the lens structure at the first state. The ball lens is adapted for modifying the geometry of the lens structure so as to adjust the effective focal power of the lens structure at the first state and the second state, respectively. In one embodiment, the effective power of the lens structure at the second state is less than the effective power of the lens structure at the first state.

In yet another aspect, the present invention relates to an accommodative intraocular lens for implantation in an eye of a living subject having a lens capsule and a lens substance contained in the lens capsule. In one embodiment, the accommodative intraocular lens includes a lens structure having a geometry and a focal power associated with the geometry, the lens geometry being changeable in response to a force applied to the lens structure, and means for engaging the lens structure with the lens capsule of the eye of the living subject such that contraction of the lens capsule pushes the lens structure contracting inwardly and relaxation of the lens capsule pulls the lens structure extending outwardly so as to change the geometry of the lens structure to adjust the focal power of the lens structure accordingly.

In one embodiment, the lens structure has an inner surface defining a volume, an outer surface, a thickness defined therebetween the inner surface and the outer surface, and an edge, where the volume of the lens structure is filled with a liquid gel. In one embodiment, the engaging means has an elastic thin wire ring. In another embodiment, the engaging means comprises a silicone rubber flat ring having a plurality of hooks. In an alternative embodiment, the engaging means comprises a plurality of ridge bars.

In a further aspect, the present invention relates to an accommodative intraocular lens for implantation in an eye of a living subject having a lens capsule and a lens substance contained in the lens capsule. In one embodiment, the accommodative intraocular lens includes a lens structure defining a volume, the volume filled with an optical transparent liquid, and a ring frame engaging the lens structure at an edge with a radius at a plurality of positions and the lens capsule at an equator at a plurality of positions.

In yet a further aspect, the present invention relates to a method of constructing an accommodative intraocular lens for implantation in an eye of a living subject having a lens capsule and a lens substance contained in the lens capsule. In one embodiment, the method includes the steps of forming a lens structure having a geometry and a focal power associated with the geometry, the lens geometry being changeable in response to a force applied to the lens structure, forming a frame, and engaging the frame with the lens structure and the lens capsule of the eye of the living subject such that contraction of the lens capsule pushes the lens structure contracting inwardly and relaxation of the lens capsule pulls the lens structure extending outwardly so as to change the lens geometry of the lens structure to adjust the focal power of the lens structure accordingly.

In one embodiment, the step of forming a lens structure comprises the step of forming a first film and a second film, each of the first film and the second film having an edge, attaching the edge of the first film to the edge of the second film to form a volume therebetween the first film and the second film, and filling a gel into the volume. In one embodiment, the first film and the second film are made of an elastic silicone rubber, where the elastic silicone rubber comprises one of an elastomeric polydimethylsiloxane and a hydrogel. The gel includes a liquid gel.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 shows a perspective view of (A) an unaccommodated lens, and (B) an accommodated lens, both of them in the prior art.

FIG. 2 shows a chart of presbyopic changes in the amplitude of accommodation due to changes with age in the lens.

FIG. 3 shows several configurations of the IOL in the prior art.

FIG. 4 shows an accommodative IOL in the prior art.

FIG. 5 shows an accommodative IOL according to one embodiment of the present invention: (A) a cross-sectional view of a lens structure in a first state, (B) a cross-sectional view of the lens structure in a second state, and (C) a top view of the accommodative IOL.

FIG. 6 shows an accommodative IOL according to another embodiment of the present invention: (A) a cross-sectional view of a lens structure in a first state, (B) a cross-sectional view of the lens structure in a second state, and (C) a top view of the accommodative IOL.

FIG. 7 shows an accommodative IOL according to an alternative embodiment of the present invention: (A) a cross-sectional view of the accommodative IOL with a lens structure in a first state, (B) a cross-sectional view of the accommodative IOL with the lens structure in a second state, and (C) a top view of the accommodative IOL.

FIG. 8 shows a cross-sectional view of the accommodative IOL according to one embodiment of the present invention.

FIG. 9 shows an accommodative IOL according to another embodiment of the present invention: (A) a cross-sectional view of the accommodative IOL with a lens structure in a first state, and (B) a cross-sectional view of the accommodative IOL with the lens structure in a second state.

FIG. 10 shows an accommodative IOL according to a different embodiment of the present invention: (A) a cross-sectional view of the accommodative IOL with a lens structure in a first state, (B) a cross-sectional view of the accommodative IOL with the lens structure in a second state, and (C) a top view of the accommodative5 IOL.

FIG. 11 shows a top view of an accommodative IOL according to an alternative embodiment of the present invention.

FIG. 12 shows schematically a process of fabricating a lens structure according to one embodiment of the present invention: (A) forming a first film and a second film by a lens fabrication station, and (B) attaching the first film to the second film to form a volume and injecting an optically transparent liquid to the volume to form a lens structure.

FIG. 13 shows a device for simulating an accommodative effect of an accommodative IOL according to one embodiment of the present invention: (A) a cross-sectional view of the device, (B) a cross-sectional view of an iris diaphragm portion of the device, and (C) a perspective view of a tweezer as shown in FIG. 13B.

FIG. 14 shows in vitro simulation of the accommodation function of an eye: (A) a diaphragm having a plurality of tweezers attached, (B) a posterior view of an animal eye clamped to the diaphragm, and (C) a anterior view of an animal eye clamped to the diaphragm.

FIG. 15 shows the in vitro simulation of the accommodation function of an eye shown in FIG. 14, by adjusting the diameter of the diaphragm: (A) and (B) the in vitro simulation results for two different diameters of the diaphragm.

FIG. 16 shows accommodative IOLs and simulation of the accommodation function of the accommodative IOLs according to one embodiment of the present invention: (A) a single Gaussian lens, (B) an accommodative IOL formed with 8 Gaussian lenses, (C) an accommodative IOL formed with 6 Gaussian lenses, (D) an customized IOL implanted into a lens capsule, (E) the IOL of FIG. 16C squeezed into a small diameter, and (F) the IOL of FIG. 16C extended into a large diameter.

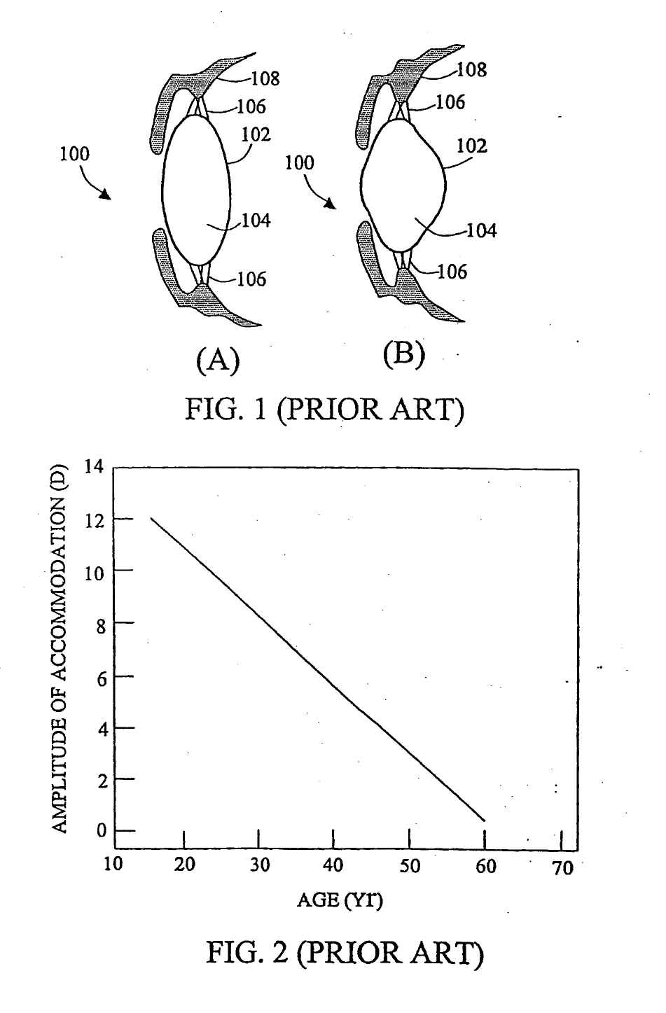

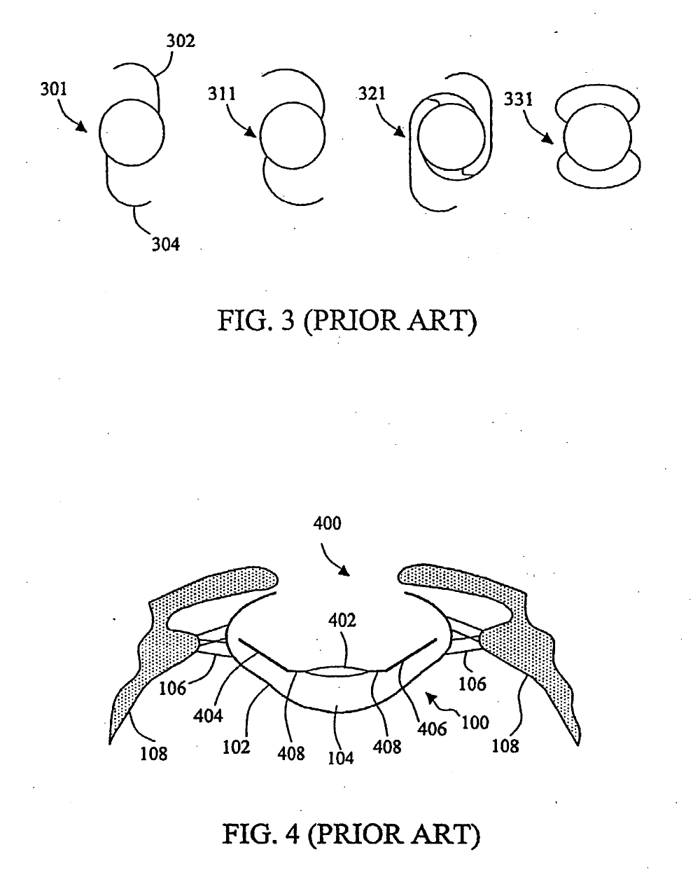

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like parts throughout the views. As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise.

The description will be made as to the embodiments of the present invention in conjunction with the accompanying drawings 1-16. In accordance with the purposes of this invention, as embodied and broadly described herein, this invention, in one aspect, relates to an accommodative IOL for implantation in an eye of a living subject having a lens capsule and a lens substance contained in the lens capsule. The living subject can be a human being or an animal. Among other things, one unique feature of the present invention is the utilization of geometrical changes of the lens capsule of the living subject to adjust a focal power of an accommodative IOL implanted in the lens capsule. In one embodiment, the accommodative IOL includes a lens structure having a geometry and a focal power associated with the geometry, where the lens geometry is changeable in response to a force applied to the lens structure, and means for engaging the lens structure with the lens capsule of the eye of the living subject such that contraction of the lens capsule pushes the lens structure contracting inwardly and relaxation of the lens capsule pulls the lens structure extending outwardly so as to change the geometry of the lens structure to adjust the focal power of the lens structure accordingly.

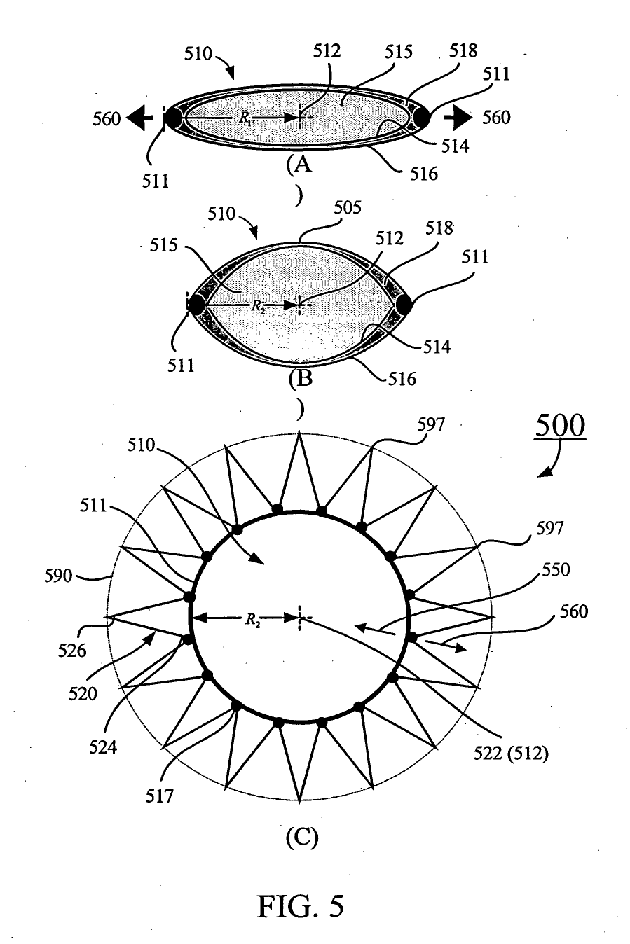

Referring in general now to FIGS. 5-9, and in particular to FIG. 5 first, an accommodative IOL 500 for implantation in an eye of a living subject in one embodiment has a lens structure 510. As shown in FIGS. 5A and 5B, the lens structure 510 has a center of geometry 512, an inner surface 514 defining a volume 515, an outer surface 516, a thickness 518 defined therebetween the inner surface 514 and the outer surface 516, and an edge 511. The lens structure 510, in one embodiment, is in the form of a lens bag that is convex. Each of the inner surface 514 and the outer surface 516 of the lens structure 510 has a variable curvature and a projected geometric configuration of a circle, and the edge 511 of the lens structure 510 is substantially circular. In the embodiment shown in FIGS. 5A and 5B, the thickness 518 of the lens structure 510 is non-uniform: the thickness at the edge 511 is thicker than the thickness at the middle 505 of the lens structure 510. The thickness 518 of the lens structure 510 can be varied or variable when the lens structure 510 is made of an elastic material and subject to an applied force. The thickness can also be uniform (not shown). In one embodiment, the volume 515 of the lens structure 510 is filled with an optically transparent liquid. The optically transparent liquid can be a liquid gel, such as a silicone gel, which has a high viscosity index, a high optical transparency and a high refractive index. Other liquid gels can also be used to practice the current invention. The lens structure 510 has an effective focal power that is associated with its geometry.

In one embodiment, the lens structure 510 is made of an elastic silicone rubber, which allows the lens structure 510 to change its geometry in response to a force applied to the lens structure and therefore adjust its effective focal power. Material like elastomeric polydimethylsiloxane (hereinafter “PDMS”), for example, Dow Coming Sylgard 184, (Dow Coming Corp., Midland, Mich.), can be used to fabricate the lens structure 510. Other material such as hydrogel, can also be employed to form the lens structure 510.

Furthermore, the accommodative IOL 500 has a frame 520. In one embodiment, as shown in FIG. 5C, the frame 520 has a center of geometry 522, a plurality of inner ends 524 and a plurality of outer ends 526, where the plurality of inner ends 524 of the frame 520 are attached to the edge 511 of the lens structure 510 at a plurality of positions 517, respectively, such that the center of geometry 522 of the frame 520 overlaps substantially with the center of geometry 512 of the lens structure 510. The plurality of outer ends 526 of the frame 520 are attached to an equator portion 590 of the lens capsule at a plurality of positions 597, respectively. The frame 520 is elastic and adapted to be in contact with and responsive to the lens capsule of the eye of the living subject. In one embodiment, the frame 520 includes an elastic thin wire ring in a shape adapted for fitting to the equator portion of the lens capsule. For the embodiment shown in FIG. 5C, the frame 520 is a closed-loop structure that has a multi inner ends 524 and outer ends 526. One advantage of the structure of the multi inner ends and outer ends is that it allows less contact between the frame 520 and the lens capsule of the eye, which may be more suitable to people having sensitive eyes, for instance. For this embodiment, the frame 520 may be considered as a closed-loop, zigzag structure.

The lens structure 510 and the frame 520 of the accommodative IOL 500 are adapted such that the lens structure 510 has a contraction force 550 directing inwardly to the center of geometry 512 of the lens structure 510 and the frame 520 has an expansion force 560 directing outwardly from the center of geometry 522 of the frame 520. When the lens capsule relaxes, the frame 520 pulls the lens structure 510 to be in a first state with an effective focal power, where the edge 511 of the lens structure 510 has a radius, R₁, as shown in FIG. 5A. When the lens capsule contracts and presses the fame 520 inwardly to the center of geometry 522 of the frame 520, the motion of the frame 520 causes the lens structure 510 to move inwardly to the center of geometry 512 of the lens structure 510 from the first state to a second state with an effective focal power that is different from the effective power of the lens structure at the first state. In the second state of the lens structure 510, as shown in FIG. 5B, the radius of the edge 511 of the lens structure 510 is R₂ that is less than R₁. As a result, the effective power of the lens structure at the second state is greater than the effective power of the lens structure at the first state, which allows the accommodative IOL 500 to be able to offer accommodation.

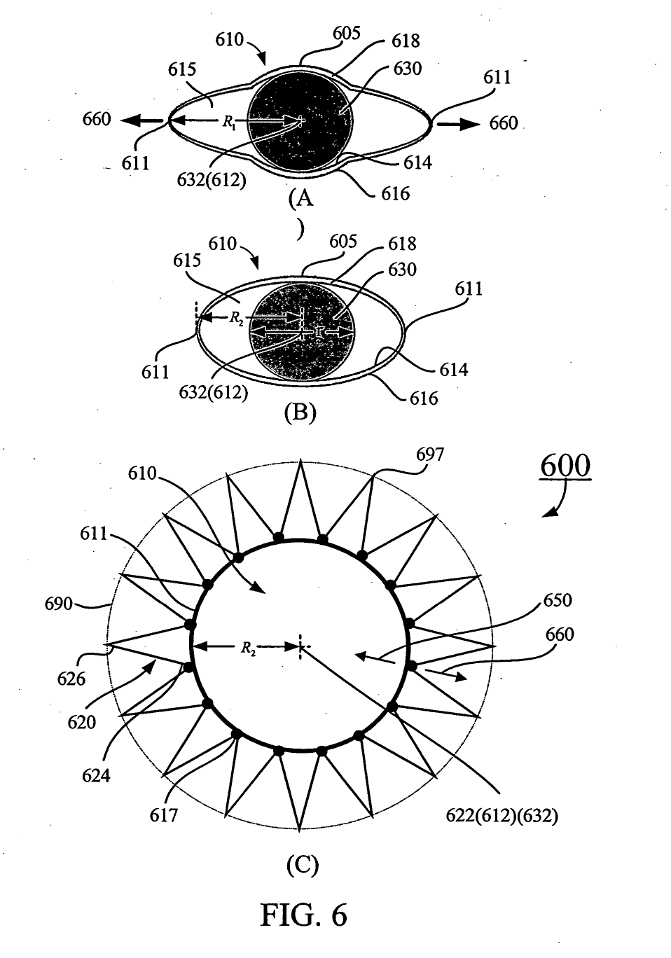

Both the lens structure 510 and the frame 520 of the accommodative IOL 500 can have various configurations. For examples, the lens structure can have different profiles and geometries. In one embodiment, the frame can be an annular or ring structure. In another embodiment, the frame can be a multi-round-cornered structure. Alternatively, the frame can be an open-loop structure. Several configurations available to the lens structure 510 and the frame 520 of accommodative IOL 500 will be discussed in more detail below in connection with embodiments of the present invention as shown in FIGS. 6-9.

Referring now to FIG. 6, an accommodative IOL 600 for implantation in the lens capsule of an eye of a living subject in one embodiment has a lens structure 610 having a center of geometry 612, an inner surface 614 defining a volume 615, an outer surface 616, a thickness 618 defined therebetween the inner surface 614 and the outer surface 616, and an edge 611. The lens structure 610 has an effective focal power that is associated with geometry of the lens structure 610. In one embodiment, the lens structure 610 is in the form of a convex lens bag, where each of the inner surface 614 and the outer surface 616 of the lens structure 610 has a variable curvature and a projected geometric configuration of a circle, and the edge 611 of the lens structure 610 is substantially circular. In the embodiment shown in FIGS. 6A and 6B, the thickness 618 of the lens structure 610 is non-uniform: the thickness 618 at the edge 611 is thicker than the thickness at the middle 605 of the lens structure 610. The thickness can also be uniform (not shown). The lens structure 610 is geometrically changeable in response to a force applied to the lens structure 610. The thickness can be variable in response to a force applied to the lens structure 610 as well.

The accommodative IOL 600 further has a ball lens 630. The ball lens 630 has a center of geometry 632 and a predetermined diameter, r, and is positioned in the volume 615 of the lens structure 610 with its center of geometry 632 substantially overlapping with the center of geometry 612 of the lens structure 610, as shown in FIGS. 6A and 6B. The ball lens 630 is a solid lens and formed with a gel. The rest of the volume 615 of the lens structure 610 is filled with an optically transparent liquid gel that is softer than the gel forming the ball lens 630. The ball lens 630 is adapted for modifying the geometry of the lens structure 610 so as to adjust the effective focal power of the lens structure 610.

Moreover, the accommodative IOL 600 has a frame 620 having a center of geometry 622, a plurality of inner ends 624 and a plurality of outer ends 626, wherein the plurality of inner ends 624 of the frame 620 are attached to the edge 611 of the lens structure 610 at a plurality of positions 617, respectively, such that the center of geometry 622 of the frame 620 overlaps substantially with the center of geometry 612 of the lens structure 610, and the plurality of outer ends 626 of the frame 620 are attached to an equator portion 690 of the lens capsule at a plurality of positions 697, respectively.

The lens structure 610 and the frame 620 of the accommodative IOL 600 are adapted such that the lens structure 610 has a contraction force 650 directing inwardly to the center of geometry 612 of the lens structure 610 and the frame 620 has an expansion force 660 directing outwardly from the center of geometry 622 of the frame 620, respectively. When the lens capsule relaxes, the frame 620 pulls the lens structure 610 to be in a first state, as shown in FIG. 6A, where the edge 611 of the lens structure 610 is sized with a radius, R₁, and the len shape of the accommodative IOL 600 is determined by the ball lens 630. When the lens capsule contracts and presses the fame 620 inwardly to the center of geometry 622 of the frame 620, the motion of the frame 620 causes the lens structure 610 to move inwardly to the center of geometry 612 of the lens structure 610 from the first state to a second state, where the radius of the edge 611 of the lens structure 610 decreases to R₂, and the lens shape of the accommodative IOL 600, which is determined by the lens structure 610, changes accordingly to the configuration as shown in FIG. 6B. Accordingly, the effective power of the lens structure at the second state is less than the effective power of the lens structure at the first state.

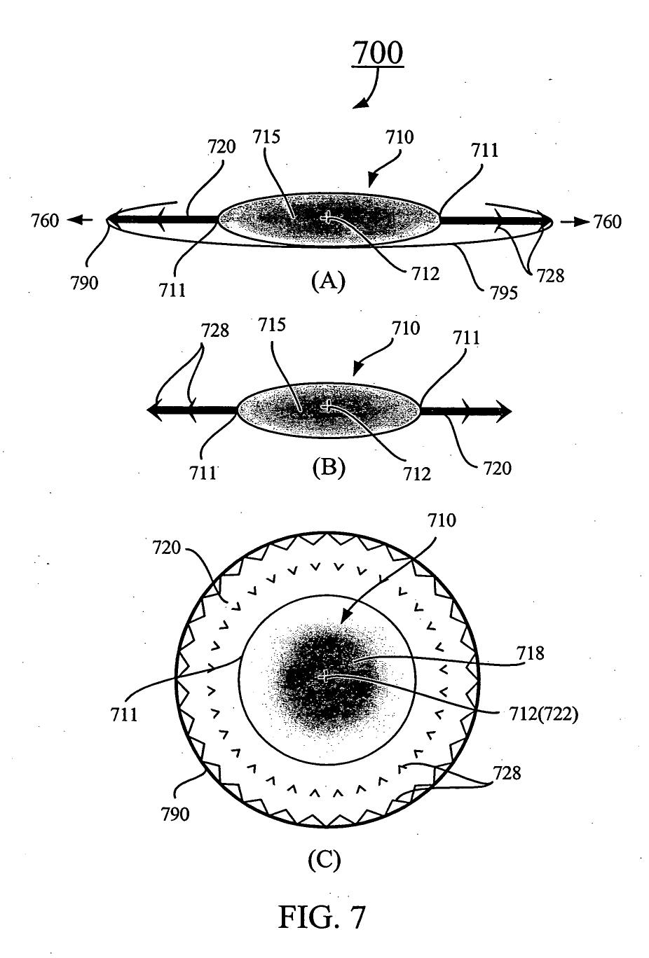

Referring to FIG. 7, an accommodative IOL 700 for implantation in an eye of a living subject having a lens capsule 795 and a lens substance contained in the lens capsule 795 is shown according to another embodiment of the present invention. In the embodiment shown in FIG. 7, the accommodative IOL 700 includes a lens structure that is in the form of a convex lens bag 710. The convex lens bag 710 defines a volume 715 that is filled with an optically transparent liquid or gel of high optical index. The convex lens bag 710 has a circular edge 711. A flat ring frame 720 extending outwardly from the circular edge 711 of the convex lens bag 710 at a predetermined shape is adapted for fitting to the lens capsule 795 and being responsive to the lens capsule 795 of the eye of the living subject, as shown in FIG. 7A. The flat ring 720 has a plurality of hooks 728 at predetermined positions. The plurality of hooks 728 of the flat ring 720 are stuck into the lens capsule 795 at the equator area 790 such that the stretching of the ciliary muscle surrounding the lens capsule 795 pulls the accommodative IOL 700 extending outwardly through the lens capsule 795 and the contraction of the ciliary muscle surrounding the lens capsule 795 pushes the accommodative IOL 700 contracting inwardly through the lens capsule 795 and therefore the radius of the edge 711 of the convex lens bag 710 is changed. Accordingly, the focal power of the convex lens bag 710 is adjusted. The flat ring frame 720 can be made of a silicone rubber, and the plurality of hooks 728 of the flat ring frame 720 can be made of relative ridged material.

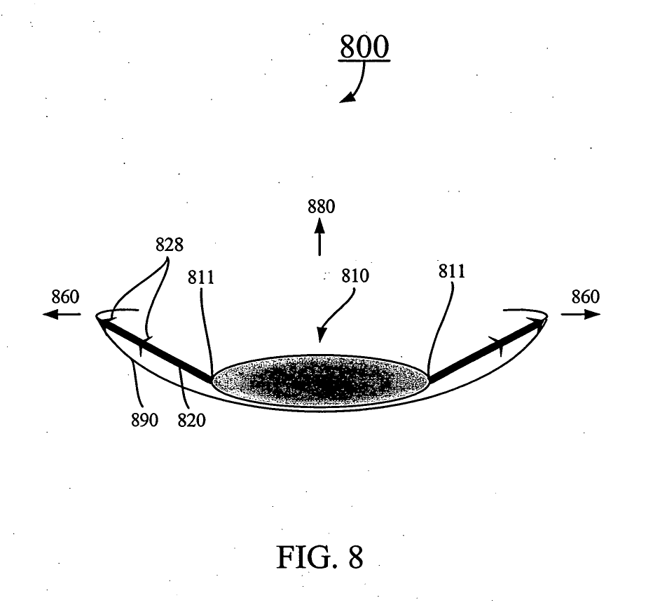

The ring frame 720 extending outwardly from the circular edge 711 of the convex lens bag 710 of the accommodative IOL 700 can be formed in a different shape. For example, in an embodiment shown in FIG. 8, a ring frame 820 of an accommodative IOL 800 is formed in a cone shape. The accommodative IOL 800 is implanted in a lens capsule of an eye of a living subject by attaching the ring frame 820 to the lens capsule. In practice, when the lens capsule 890 of an eye of a living subject is stretched outwardly in direction 860 into a bigger diameter, the lens bag 810 will be pulled forward in direction 880. As a result, a distance between the accommodative IOL 800 and an object (not shown here) to be focused is changed, and therefore the effective focal power of the accommodative IOL 800 is adjusted accordingly.

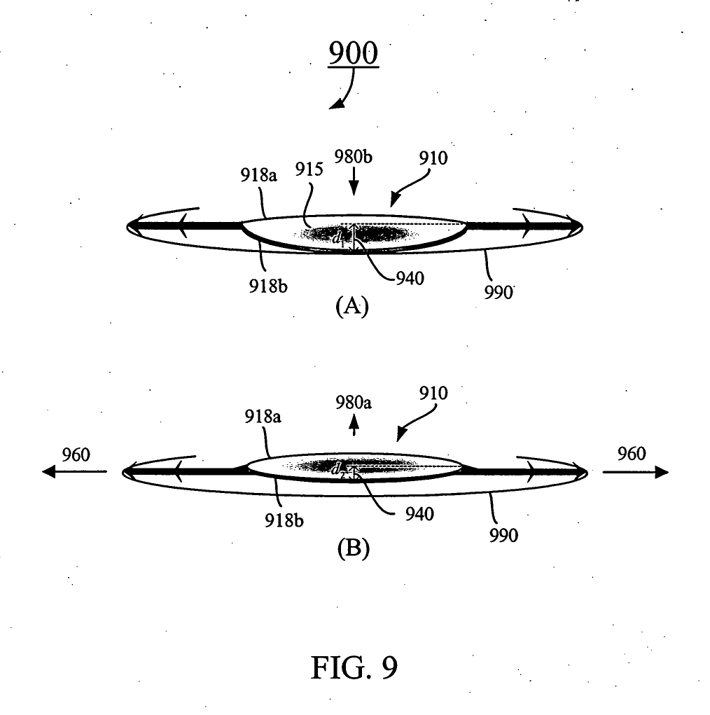

FIG. 9 shows an another embodiment of an accommodative IOL 900, where a silicone lens bag 910 has an anterior wall 918a and a posterior wall 918b defining a volume 915, and the anterior wall 918a and the posterior wall 918b are formed in different profiles, in which the posterior wall 918b of the silicone lens bag 910 is curved, the anterior wall 918a of the silicone lens bag 910 is flat, and the posterior wall 918b of the silicone lens bag 910 is thicker than the anterior wall 918a of the silicone lens bag 910. The lens bag 910 has a thickness 940, d₁, defined therebetween a center of the anterior wall 918a and a center of the posterior wall 918b. As shown in FIG. 9A, when the silicone lens bag 910 is at its smaller diameter, both the anterior wall 918a and the posterior wall 918b of the silicone lens bag 910 tend toward posterior in direction 980b. The silicone lens bag 910 is thicker and has an effective focus power. When the ciliary muscle surrounding the lens capsule 995 of the eye pulls the lens capsule 995, the movement of the lens capsule 995 in direction 960 causes the accommodative IOL 900 implanted into the lens capsule 995 to extend into a bigger diameter. Accordingly, both the anterior wall 918a and the posterior wall 918b of the silicone lens bag 910 move forward in direction 980a. The posterior wall 918b of the silicone lens bag 910 becomes flatter, while the anterior wall 918a of the silicone lens bag 910 becomes convex. The thickness 940 of the silicone lens bag 910 for this state d₂, is less than d₁, as shown in FIG. 9B. The silicone lens bag 910 has a lower focal power in this state shown in FIG. 9B than that of the state shown in FIG. 9A.

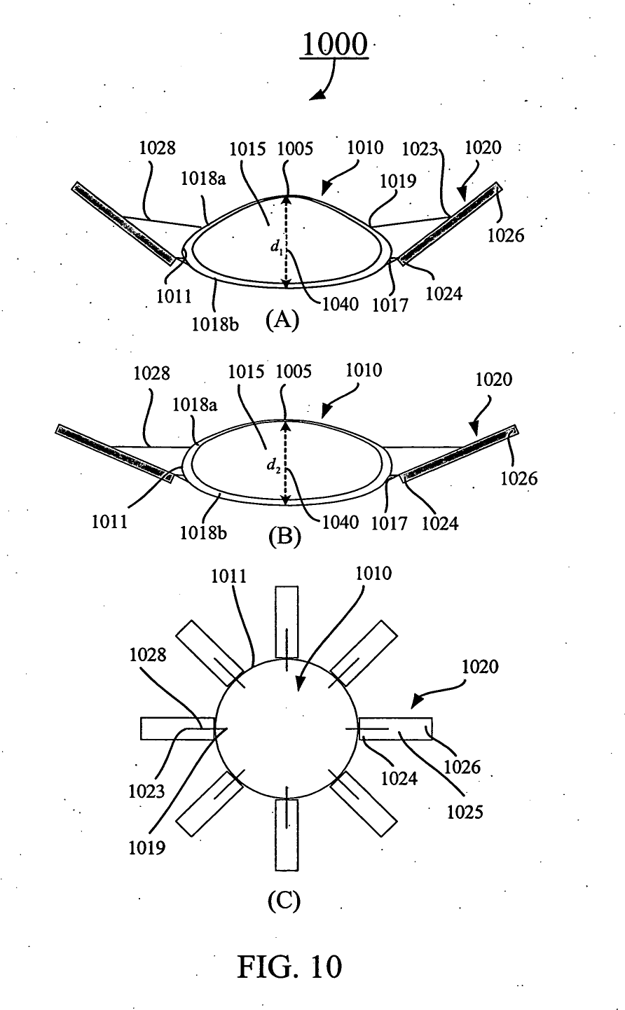

FIG. 10 shows an alternative embodiment of an accommodative IOL 1000 for implantation in an eye of a living subject having a lens capsule. The accommodative IOL 1000 is in the form of a lens bag 1010 with an effective focal power associated with geometry of the lens bag 1010 and a frame 1020 for engaging the lens beg 1010 with the lens capsule of the eye of the living subject. As shown in FIGS. 10A and 10B, the lens bag 1010 has an anterior wall 1018a and a posterior wall 1018b defining a volume 1015 and an edge 1011. The lens bag 1010 has a thickness 1040 defined therebetween a center of the anterior wall 1018a and a center of the posterior wall 1018b. Each of the anterior wall 1018a and the posterior wall 1018b has a variable curvature. The variable curvature of the anterior wall 1018a is substantially different from that of the posterior wall 1018b. The edge 1011 of the lens bag is substantially circular. The volume 1015 of the lens bag is filled with an optically transparent liquid such as a liquid gel. In operation, the geometry of the lens bag 1010 can be changed from one state to another state in response to a force applied to the lens bag 1010. When the lens bag 1010 is in a condition-free state, as shown in FIG. 10A, the curvature of the anterior wall 1018a is in its maximum value, and the lens bag 1010 has a maximum thickness, d₁. Consequently, the lens bag 1010 in the condition-free state has the highest effective focal power. The frame 1020 includes a plurality of ridge bars 1025. As shown in FIG. 10C, the frame 1020 has 10 ridge bars and is formed with a structure that is symmetrical to a center of geometry of the lens bag 1010. Other numbers of the ridge bars can also be used to practice the current invention. Each ridge bar 1025 has a first end 1024 and an opposite, second end 1026. The first end 1024 of each ridge bar 1025 is attached to the edge 1011 of the lens bag at a predetermined position by an elbow 1017. Each ridge bar 1025 is also coupled to the anterior wall 1018a of the lens bag by a string 1028 to connect the ridge bar at a point 1023 between the first end 1024 and the second end 1026 of the ridge bar 1025 to the anterior wall 1018a at a predetermined position 1019. The accommodative IOL 1000 is implanted in an eye of a living subject by associating the plurality of ridge bars 1025 of the frame 1020 to the lens capsule of the eye of the living subject. When the ciliary muscle surrounding the lens capsule of the eye of the living subject contracts, the lens capsule will push the frame 1020 of the accommodative IOL 1000 inwardly to keep the lens bag 1010 of the accommodative IOL 1000 in a first state that is the condition-free state, as shown in FIG. 10A. When the ciliary muscle surrounding the lens capsule of the eye of the living subject relaxes, the lens capsule will stretch the anterior wall 1018a of the lens bag 1010 of the accommodative IOL 1000 extending outwardly through the frame 1020 of the accommodative IOL 1000 to change the geometry of the lens bag 1010 from the first state in a second state, where the curvature of the anterior wall 1018a decreases, and the thickness 1040 of the lens bag 1010 decreases to d₂, as shown in FIG. 10B. Accordingly, the effective focal power of the lens bag 1010 in the second state and therefore the focal power of the accommodative IOL 1000 decreases.

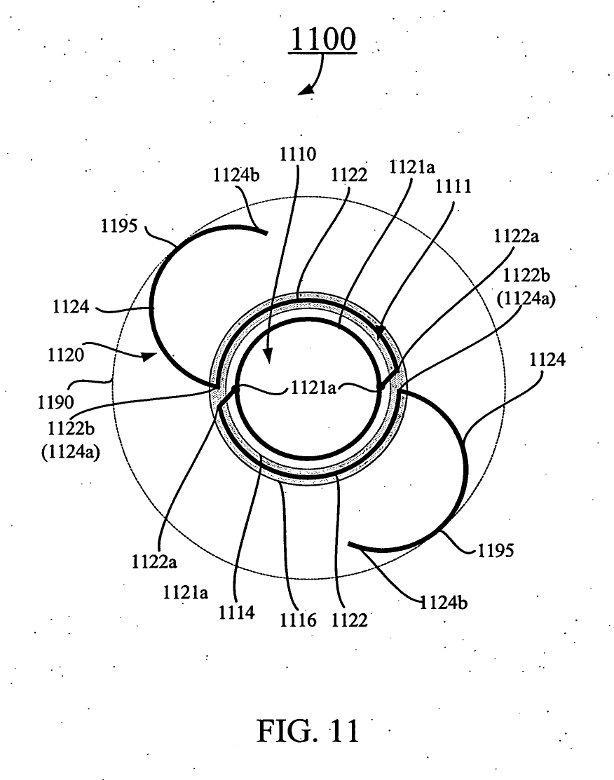

Referring now to FIG. 11, an accommodative IOL 1100 for implantation in an eye of a living subject having a lens capsule 1190 is shown according to another embodiment of the present invention. As shown in FIG. 11, the accommodative IOL includes a silicone lens bag 1110 and an elastic wire frame 1120 that is adapted for engaging the silicone lens bag 1110 with the lens capsule. The silicone lens bag 1110 includes a circular edge portion 1111 having an inner diameter 1114 and an outer diameter 1116. The elastic wire frame 1120 has a solid circle ring 1121 located inside the silicone lens bag 1110 at a position close to the inner diameter 1114 of the circular edge portion 1111. The elastic wire frame 1120 also has two half-circle rings 1122 and two haptics 1124. Each half-circle rings 1122 has a first end 1122a and a second end 1122b, respectively, and is embedded into a position between the inner diameter 1114 and the outer diameter 1116 of the edge portion 1111 with the first end 1122a welded to the solid circle ring 1121 at position 1121a. The two half-circle rings 1122 are configured in a nearly closed circle, as shown in FIG. 11. Each haptics has a first end 1124 and a second end 1124, respectively. The second end 1122b of a half-circle ring 1122 is connected to the first end 1124a of a haptics 1124. The haptics 1124 is adapted for contacting with and responding to the lens capsule 1190. In operation, when the haptics 1124 are pushed inwardly, the half circle rings 1122 will be squeezed into a smaller diameter. The half circle rings 1122 are embedded into the edge portion 1111 of the silicone lens bag 1110 under this squeezed condition. After the pressure is released, the half circle rings 1122 will tend to expend into a bigger diameter, and force the silicone lens bag 1110 to be in a state having a lower focal power. After the accommodative IOL is implanted into the lens capsule of the eye of the living subject, the contraction of the lens capsule 1190, caused by constriction of the ciliary muscle, will be able to squeeze the two half-circle rings 1122 into a smaller diameter according to the constriction force. The smaller diameter of the silicone lens has a higher focal power.

In another aspect, the present invention relates to a method of constructing an accommodative intraocular lens for implantation in an eye of a living subject having a lens capsule and a lens substance contained in the lens capsule. In one embodiment, the method includes the following steps: at first, a lens structure is formed to have a geometry and a focal power associated with the geometry. The lens geometry is changeable in response to a force applied to the lens structure. Second, a frame is formed. And then the frame is engaged with the lens structure and the lens capsule of the eye of the living subject such that contraction of the lens capsule pushes the lens structure contracting inwardly and relaxation of the lens capsule pulls the lens structure extending outwardly so as to change the lens geometry of the lens structure to adjust the focal power of the lens structure accordingly.

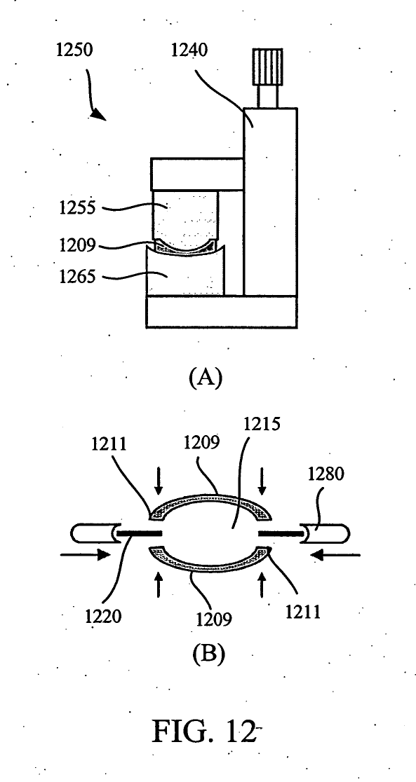

In operation, the lens structure can be fabricated as a convex lens bag. Material like PDMS can be used. The PDMS is preferably chosen because of its optical transparency, strength, and ability to be easily molded into various shapes and peeled from a surface. Referring to FIG. 12, in one embodiment, the convex lens bag can be constructed by a lens fabrication station 1250 having a micrometer adjustable stage 1240, a convex lens mold 1255 and a concave lens mold 1265 complementarily placed below the convex lens mold 1255, as shown in FIG. 12A. To form a lens film 1209, the PDMS and cure agent are poured onto a surface of the concave lens mold 1265 and, as the polymer cures, a constant pressure is applied by a convex lens mold 1255 that is controlled with a micrometer-adjustable stage 1240. After peeling the PDMS lens film 1209 from the mold, biocompatibility is imparted to the surface of the lens film 1209 with a protein-resistant molecular film prepared from an oligo(ethylene glycol)-terminated alkyltrichlorosilane (HO(CH₂CH₂O)₃SiCl₃) [23].

In one embodiment, a first and second PDMS lens films 1209 prepared in this manner are combined to form the lens bag. The zigzag elastic ring frame 1220 is squeezed into a smaller diameter, as shown in FIG. 12B. Under this condition, the first PDMS lens film 1209 and the second PDMS lens film 1209 are glued in the edge portion 1211 to form a volume 1215 therebetween the first PDMS lens film 1209 and the second PDMS lens film 1209, and embed the inner side of the ring frame in the equator of the silicone rubber bag. Silicone gel 1280 selected for its viscosity, optical clarity, and high refractive index then is injected into the lens bag.

Polypropylene, or polyamide or steel can be utilized as the elastic frame material. Medical grade epoxy can be used to glue the lenses to the elastic frame. Heat compressing can also be utilized to couple the lenses to the frame. Moreover, different or alternate frame configurations can be designed and utilized to couple the lenses to the frame.

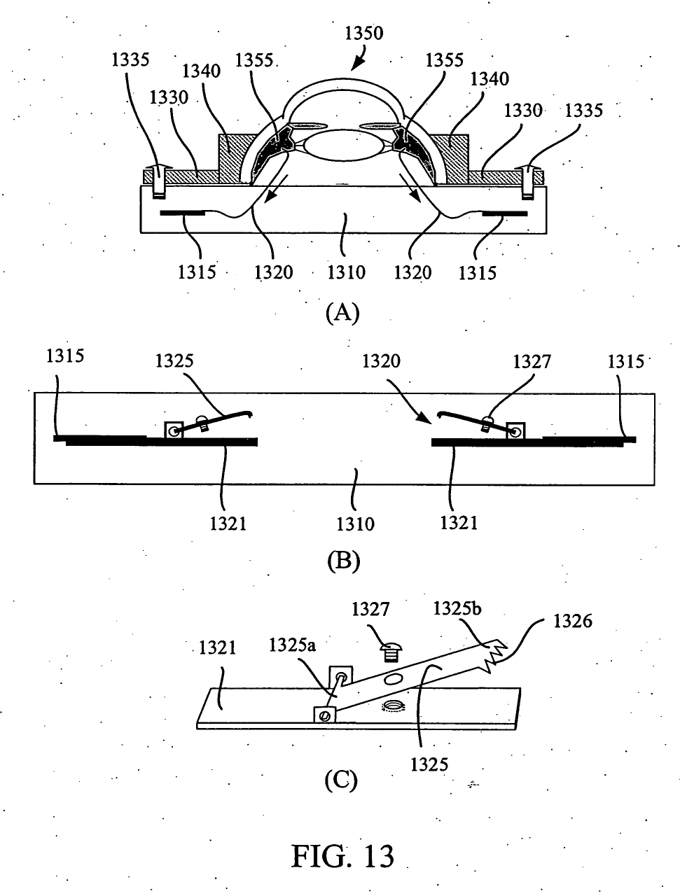

Now referring to FIGS. 13, a device for simulating an accommodative effect of an IOL is shown. In one embodiment of the present invention, the device 1300 includes an iris diaphragm 1310 having a plurality of leaves 1315. A diameter of the iris diaphragm 1310 is adjustable. The device 1300 further includes a holder 1340. The holder 1340 is mounted on the iris diaphragm 1310 for holding the accommodative IOL 1350. Moreover, the device 1300 includes a plurality of connectors 1320 that is adapted for engaging the plurality of leaves 1315 of the iris diaphragm 1310 with the accommodative IOL 1350 through the ciliary muscle 1355. Additionally, the device 1300 includes a ring 1330 that is slipped on outside of the holder 1340 and mounted in the iris diaphragm 1310 for adjusting the diameter of the iris diaphragm 1310. By adjusting the diameter of the iris diaphragm 1310, the accommodative IOL 1350 expands or contracts through pulling or pushing the connector 1320. Accordingly, the focal power of the accommodative IOL 1350 is changed. The device 1300 effectively simulates the movement of a ciliary muscle of the lens capsule.

The ring 1330 can be made of a metal or plastic. The holder 1340 can be made of a metal or plastic. The connector 1320 may includes at least one metal wire or tweezers 1320, or like. FIGS. 13A and 13B show a device having tweezers connectors. Each tweezers 1320 has a base piece 1321 being welded to one of the plurality of leaves 1315 of the iris diaphragm 1310 and a top piece 1325 with a first end 1325a mounted on the base piece 1321 and an opposite second end 1325b having a plurality of teeth 1326 for engaging with the accommodative IOL. The tweezers 1320 also includes a knob 1327 on the top piece 1325 for adjusting the engagement of the plurality of teeth 1326 with the accommodative IOL.

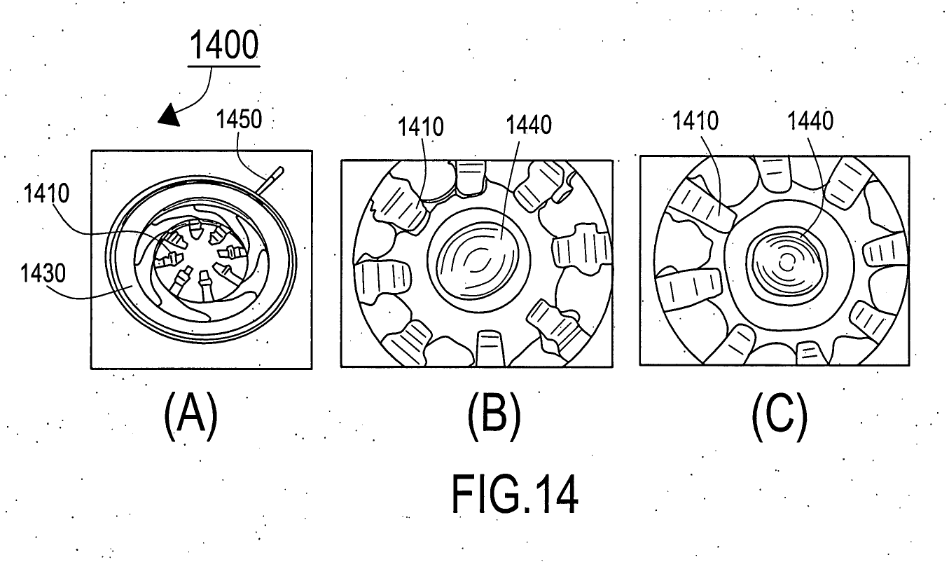

FIG. 14 shows a device for simulating an accommodative effect of an intraocular lens according to another embodiment of the present invention. The device has a plurality of tweezers 1410 attached to the leaves of an adjustable iris diaphragm 1430. A diameter of the iris diaphragm 1430 can be adjusted by an adjust member 1450. In this embodiment shown in FIG. 14A, eight tweezers are used. Other number of tweezers can also be used to practice the current invention. In vitro simulation of the accommodation function of an accommodative IOL was conducted by using a fresh animal cadaver eye. The fresh animal cadaver eye 1440 was dissected so as to obtain the lens with iris and ciliary muscle together. The ciliary muscle was clamped to the eight tweezers 1450 symmetrically, as shown in FIGS. 14B and 14C. Adjusting the diameter of the iris diaphragm 1430 causes the diameter change of the ciliary muscle and the diameter change of the lens capsule as well. This would simulate the in vivo accommodative function of the eye in an ex vivo model.

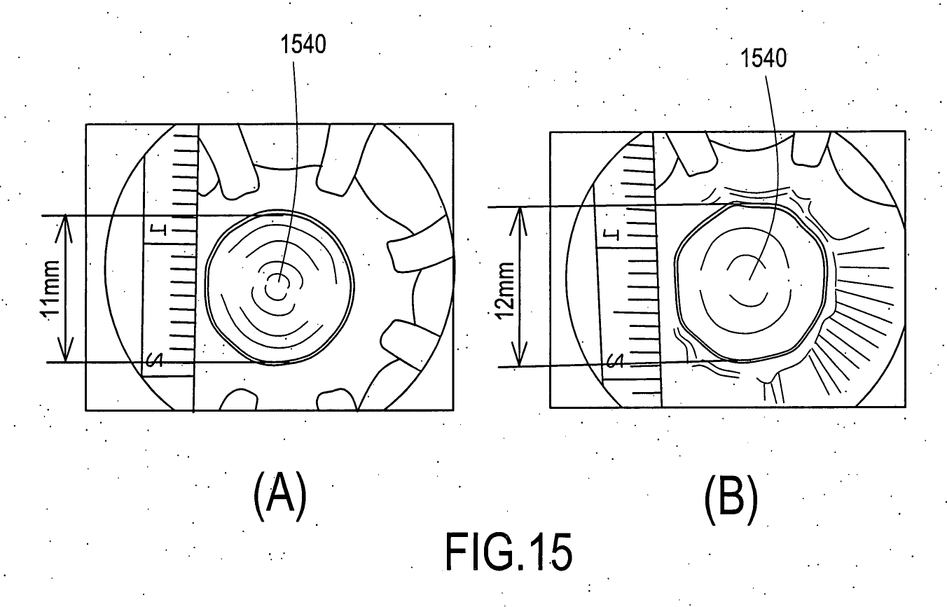

When the diameter of the adjustable diaphragm increased, it would pull the ciliary muscle outwardly. The diameter of the animal lens would be increased. In the example shown in FIG. 15, the diameter of the lens 1540 was increased from 11 mm, as shown in FIG. 15A, to 12 mm, as shown in FIG. 15B, when the diameter of the circle of tweezers changed from 17 mm to 20 mm. At the same time, the curvature of the lens anterior and posterior portions of the animal lens 1540 changed, and the animal lens 1540 was pulled to move backwards axially.

In order to measure diopter changes of the animal lens, an artificial ocular structure was assembled (not shown here). A plastic Plano-convex lens was used as cornea surface. A 3 mm diameter hole was used as pupil. The animal lens held by the adjustable device shown in FIG. 14A was set at a predetermined position beneath the cornea. A flat white surface with certain blood vessel drawing served as retina and was set at a certain distance beneath the device. Everything was screwed together so that the distance between the cornea and the retina would not change. BSS solution was filled in the artificial ocular structure. The diopter change caused by the animal lens was measured by a vertically setup autorefractor (model MRK-2000, Huvitz Co. Ltd., Gyeonggido, Korea). This artificial ocular structure was not according to human or any animal eye. It was only to set up an ocular structure, so that the refraction change can be measured by the autorefractor. Table 2 shows the diopter changes of a pig eye lens versus the diameter of the adjustable diaphragm. The diopter reading first became smaller, indicating that the focal power of the lens increased, and then became higher, indicating that the focal power of the lens decreased.

TABLE 2
Measurement of the diopter change of a pig eye lens versus the
diameter of the adjustable diaphragm.

| | Diameter of the Circle of | | | |
|---|---|---|---|---|
| | the Tweezers (mm) | | | |
| | 14 | 15 | 16 | 17 |
| Diopter Reading | +2.25 | +1.50 | +1.25 | +3.75 |

Capsulorrhexis was performed on the anterior surface of the clamped pig eye lenses. Lens content was removed. Accommodative IOLs with different ring frames were implanted into the lens capsule. The movement of the accommodative IOL responsive to diameter changes of the ciliary muscle was studied.

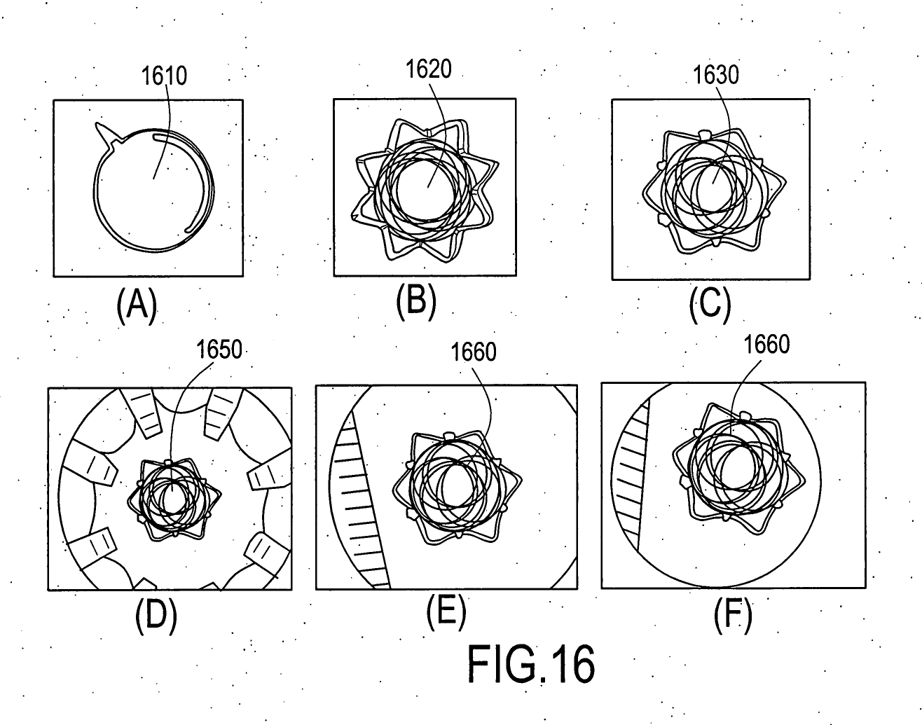

The different accommodative IOLs according to embodiments of the present invention are shown in FIGS. 16A-16C. Gaussian lenses 1610, 1620 and 1630 were made by an injection mold method from Polymer Optics, LLC, Santa Rosa, Calif. The material formed these accommodative IOLs was cyclic-olefin copolymer (COC). Other material can also be used to practice the present invention. The diameter of the single Gaussian lens 1610 was 5.5 mm, as shown in FIG. 16A. The thinnest thickness of the lens that the company could produce is 160 μm to 170 μm, versus a designed thickness of 100 μm. The accommodative IOL of 8 Gaussian lenses had a thickness of 1.6 mm, as shown in FIG. 16B, and the accommodative IOL of 6 Gaussian lenses had a thickness of 1.2 mm, as shown in FIG. 16C. Different materials with different thicknesses were used to make the zigzag frame, which included: transparency film of 100 μm thickness, PMMA film of 50 μm thickness, COC film of 60 μm and 100 μm, plastic film of 40 μm thickness, plastic film of 20 μn thickness.

Measurement of the diopter changes of the accommodative IOL by using artificial ocular setting and the autorefractor was showed in Table 3. The accommodative IOL used to perform the capsulorrhexis was formed with 6 Gaussian lenses and a frame of 20 μm thickness. As shown in FIG. 16E, when the diaphragm was adjusted to a smaller diameter, the diameter of the accommodative IOL 1660 was squeezed into a smaller diameter. Accordingly, the diopter of the accommodative IOL 1660 decreased and the diopter reading was in small values, as indicated by the second column of Table 3. In this situation, the accommodative IOL 1660 had a higher focal power. When the diaphragm was adjusted to a bigger diameter, the diameter of the accommodative IOL 1660 is extended. Consequently, the diopter of the accommodative IOL 1660 increased and the diopter reading was in large values.

The focal power of the accommodative IOL 1660 decreased. As shown in FIG. 16F, the diameter of the accommodative IOL 1660 was 0.5 mm larger than that of FIG. 16E. The diopter for the large diameter accommodative IOL 1660 increased, and the corresponding diopter reading was listed in the third column of Table 3. The diopter changes for the accommodative IOL 1660 in the above two different diameters were presented in the fourth column of Table 3.

TABLE 3
Measurement of diopter changes of an accommodative IOL by using an
artificial ocular and an autorefractor.

| | Diopter Reading for | Diopter Reading for | |
|---|---|---|---|
| Number of | Small Diameter of the | Bigger Diameter of the | Diopter |
| Test | Diaphragm | Diaphragm | Changes |
| 1 | +9.00 | +14.75 | +5.75 |
| 2 | +9.75 | +10.00 | +0.25 |
| 3 | +10.25 | +11.25 | +1.00 |

For implantation of the accommodative IOL into the cadaver animal eye in a surgical mode, cadaver sheep eye was used. An opening of 7 mm was made at the limbus. Healon was used to extend the anterior chamber. Capsulorrhexis of about 6 to 6.5 mm was made. Because the zigzag ring frame was very soft, the accommodative IOL was slide easily through the opening and implant into the capsule.

In the present invention, among other things, an accommodative IOL for implantation in an eye of a living subject is disclosed, which includes a lens structure having a geometry and a focal power associated with the geometry and a frame for engaging the lens structure with the lens capsule of the eye of the living subject such that contraction of the lens capsule pushes the lens structure contracting inwardly and relaxation of the lens capsule pulls the lens structure extending outwardly so as to change the geometry of the lens structure to adjust the focal power of the lens structure accordingly.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

References List

[1] von Helmholtz. Hb. D. Physiol. Optik., Leipzig, 1(1856); 2nd ed., Hamburg, 175 (1896).
[2] Sir Stewart Duke-Elder, and David Abrams. System of Ophthalmology, Vol V, Ophthalmic Optics and Rferaction. St. Louis, The C.V. Mosby Company. 1970. P177.
[3] Fincham. Brit. J. Ophthal., 35, 381 (1951); J. Physiol., 128, 99 (1955); Vision Res., 1,425 (1962).
[4] Fisher R F. The significance of the shape of the lens and capsular energy changes in accommodation. J. Physiol. (1969), 201, pp.21-47.
[5] Denham D, Parel J-M, Method For Ex-vivo Assessment Of Accommodation Forces, Invest Ophthalmol Vis Sci 43(Suppl): 403. 2002.
[6] Hara T, Hara T, Yasuda A, Mizumoto Y,Yamada Y. Accommodative intraocular lens with spring action. Part 2. Fixation in the living rabbit. Ophthalmic Surg. 1992; 23:632-635.
[7] Kessler J. Experiments in refilling the lens. Arch Ophthalmol 1964; 71:412-7.
[8] E. Haefliger, J-M. Parel, F. Fantes, E. W. D. Norton, D. R. Anderson, R. K.Forster, E. Hernandez, W. J. Feuer. Accommodation of an endocapsular silicone lens (phaco-ersatz) in the nonhuman primate. Opthalmology 94:471-477,1987.
[9] 0. Nishi, K. Nishi, C. Mano, M. Ichihara, T. Honda. Controlling the capsular shape in lens refilling. Arch Ophthalmol. 1997; 115:507-510.
[10] Y. Sakka, T. Hara, Y.Yamada, T.Hara, F.Hayashi. Accommodation in primate eyes after implantation of refilled endocapsular balloon. American Journal of Ophthalmology 121:210-212, 1996.
[11] Keates R H, Pearce J L, Schneider R T: Clinical results of the multifocal lens. J Cataract Refract Surg 13:557-560, 1987.
[12] Percival P. Indications for the multizone bifocal implant. J Cataract Refract Surg 16:193-197,1990.
[13] Jacobi P C, Konen W. Effect of age and astigmatism on the AMO Array multifocal intraocular lens. J Cataract Refract Surg 21:556-561,1995.
[14] Christie B, Nordan L, Chipman R, Gupta A. Optical performance of an aspheric multifocal intraoclar lens. J Cataract Refract Surg 17:583-591,1991.
[15] Bellucci R, Giardini P. Pseudoaccommodation with the 3M diffractive multifocal intraocular lens:A refraction study of 52 subjects. J Cataract Refract Surg 19:32-35,1993.
[16] Holladay J T, van Dijk H, Lang A, et al. Optical perfomance of multifocal intraocular lenses. J Cataract Refract Surg 1990; 16:413-422.
[17] Olsen T, Corydon L. Contrast sensitivity in patients with a new type of multifocal intraocular lens. J Cataract Refract Surg 16:42-46, 1990.
[18] Auffarth G U, Hunold W, Wesendahl T A, Mehdom E. Depth of focus and functional results in patients with multifocal intraocular lenses: A long-term follow-up. J Cataract Refract Surg 19:685-689,1993.
[19] Hara T, Hara T, Yasuda A, Yamada Y. Accommodative intraocular lens with spring action. Part 1. Design and placement in an excised animal eye. Ophthalmic Surg. 1990; 21:128-133.
[20] Cumming J S, Kammann J. Experience with an accommodating IOL. J Cataract Refract Surg 1996; 22:1001.
[21] Samalonis L, Making adjustments after implantation. Eye World 2000; 5:54.
[22] Shen J H, O'Day DM: Designing of an Accommodative Intraocular Lens. Invest Ophthalmol Vis Sci 43(Suppl):402. 2002.
[23] Lee S W, Laibinis PE Protein-resistant coatings for glass and metal oxide surfaces derived from oligo(ethylene glycol)-terminated alkyltrichlorosilanes. Biomaterials19:1669-1675, 1998.

1. An accommodative intraocular lens for implantation in an eye of a living subject having a lens capsule and a lens substance contained in the lens capsule, comprising:
a. a lens structure having a center of geometry, an inner surface defining a volume, an outer surface, a thickness defined therebetween the inner surface and the outer surface, and an edge, the volume filled with an optically transparent liquid; and
b. a frame having a center of geometry, a plurality of inner ends and a plurality of outer ends,
wherein the plurality of inner ends of the frame are attached to the edge of the lens structure at a plurality of positions, respectively, such that the center of geometry of the frame overlaps substantially with the center of geometry of the lens structure, and the plurality of outer ends of the frame are attached to an equator portion of the lens capsule at a plurality of positions, respectively.

2. The accommodative intraocular lens of claim 1, wherein the lens structure and the frame are adapted such that the lens structure has a contraction force directing inwardly to the center of geometry of the lens structure and the frame has an expansion force directing outwardly from the center of geometry of the frame, and when the lens capsule relaxes, the frame pulls the lens structure to be in a first state with an effective focal power, and when the lens capsule contracts and presses the fame inwardly to the center of geometry of the frame, the motion of the frame causes the lens structure to move inwardly to the center of geometry of the lens structure from the first state to a second state with an effective focal power that is different from the effective power of the lens structure at the first state.

3. The accommodative intraocular lens of claim 2, wherein the effective power of the lens structure at the second state is greater than the effective power of the lens structure at the first state.

4. The accommodative intraocular lens of claim 3, wherein the lens structure is convex.

5. The accommodative intraocular lens of claim 4, wherein the edge of the lens structure is substantially circular.

6. The accommodative intraocular lens of claim 5, wherein each of the inner surface and the outer surface of the lens structure has a variable curvature and a projected geometric configuration of a circle.

7. The accommodative intraocular lens of claim 6, wherein the thickness of the lens structure is uniform.

8. The accommodative intraocular lens of claim 6, wherein the thickness of the lens structure is non-uniform.

9. The accommodative intraocular lens of claim 3, wherein the lens structure is made of an elastic silicone rubber.

10. The accommodative intraocular lens of claim 9, wherein the elastic silicone rubber comprises one of an elastomeric polydimethylsiloxane and a hydrogel.

11. The accommodative intraocular lens of claim 1, wherein the frame comprises a structure that is symmetrical to the center of geometry of the frame.

12. The accommodative intraocular lens of claim 11, wherein the frame comprises a closed-loop structure.

13. The accommodative intraocular lens of claim 12, wherein the frame comprises an elastic thin wire ring in a shape adapted for fitting to the equator portion of the lens capsule.

14. The accommodative intraocular lens of claim 11, wherein the frame comprises an open-loop structure.

15. The accommodative intraocular lens of claim 1, wherein the optically transparent liquid comprises a liquid gel.

16. An accommodative intraocular lens for implantation in an eye of a living subject having a lens capsule and a lens substance contained in the lens capsule, comprising:
a. a lens structure having a center of geometry, an inner surface defining a volume, an outer surface, a thickness defined therebetween the inner surface and the outer surface, and an edge;
b. a ball lens having a center of geometry and a predetermined diameter, r, and positioned in the volume of the lens structure with its center of geometry substantially overlapping with the center of geometry of the lens structure, wherein the rest of the volume is filled with a first gel; and
c. a frame having a center of geometry, a plurality of inner ends and a plurality of outer ends,
wherein the plurality of inner ends of the frame are attached to the edge of the lens structure at a plurality of positions, respectively, such that the center of geometry of the frame overlaps substantially with the center of geometry of the lens structure, and the plurality of outer ends of the frame are attached to an equator portion of the lens capsule at a plurality of positions, respectively.

17. The accommodative intraocular lens of claim 16, wherein the lens structure and the frame are adapted such that the lens structure has a contraction force directing inwardly to the center of geometry of the lens structure and the frame has an expansion force directing outwardly from the center of geometry of the frame, and when the lens capsule relaxes, the frame pulls the lens structure to be in a first state with an effective focal power, and when the lens capsule contracts and presses the fame inwardly to the center of geometry of the frame, the motion of the frame causes the lens structure to move inwardly to the center of geometry of the lens structure from the first state to a second state with an effective focal power that is different from the effective power of the lens structure at the first state.

18. The accommodative intraocular lens of claim 17, wherein the ball lens is adapted for modifying the geometry of the lens structure so as to adjust the effective focal power of the lens structure at the first state and the second state, respectively.

19. The accommodative intraocular lens of claim 18, wherein the effective power of the lens structure at the second state is less than the effective power of the lens structure at the first state.

20. The accommodative intraocular lens of claim 19, wherein the lens structure is convex.

21. The accommodative intraocular lens of claim 20, wherein the edge of the lens structure is substantially circular.

22. The accommodative intraocular lens of claim 21, wherein each of the inner surface and the outer surface of the lens structure has a variable curvature and a projected geometric configuration of a circle.

23. The accommodative intraocular lens of claim 22, wherein the thickness of the lens structure is uniform.

24. The accommodative intraocular lens of claim 23, wherein the thickness of the lens structure is non-uniform.

25. The accommodative intraocular lens of claim 19, wherein the lens structure is made of an elastic silicone rubber.

26. The accommodative intraocular lens of claim 25, wherein the elastic silicone rubber comprises one of an elastomeric polydimethylsiloxane and a hydrogel.

27. The accommodative intraocular lens of claim 16, wherein the frame comprises a structure that is symmetrical to the center of geometry of the frame.

28. The accommodative intraocular lens of claim 27, wherein the frame comprises a closed-loop structure.

29. The accommodative intraocular lens of claim 28, wherein the frame comprises an elastic thin wire ring in a shape adapted for fitting to the equator portion of the lens capsule.

30. The accommodative intraocular lens of claim 27, wherein the frame comprises an open-loop structure.

31. The accommodative intraocular lens of claim 16, wherein the ball lens comprises a solid lens.

32. The accommodative intraocular lens of claim 16, wherein the ball lens is formed with a second gel that is harder than the first gel.

33. The accommodative intraocular lens of claim 16, wherein the first gel comprises an optically transparent liquid gel.

34. An accommodative intraocular lens for implantation in an eye of a living subject having a lens capsule and a lens substance contained in the lens capsule, comprising:
a. a lens structure having a geometry and a focal power associated with the geometry, the lens geometry being changeable in response to a force applied to the lens structure; and
b. means for engaging the lens structure with the lens capsule of the eye of the living subject such that contraction of the lens capsule pushes the lens structure contracting inwardly and relaxation of the lens capsule pulls the lens structure extending outwardly so as to change the geometry of the lens structure to adjust the focal power of the lens structure accordingly.

35. The accommodative intraocular lens of claim 34, wherein the lens structure has an inner surface defining a volume, an outer surface, a thickness defined therebetween the inner surface and the outer surface, and an edge.

36. The accommodative intraocular lens of claim 35, wherein the volume of the lens structure is filled with a liquid gel.

37. The accommodative intraocular lens of claim 35, wherein the edge of the lens structure is substantially circular.

38. The accommodative intraocular lens of claim 37, wherein the lens structure is convex.

39. The accommodative intraocular lens of claim 38, wherein each of the inner surface and the outer surface of the lens structure has a variable curvature and a projected geometric configuration of a circle.

40. The accommodative intraocular lens of claim 34, wherein the engaging means comprises an elastic thin wire ring.

41. The accommodative intraocular lens of claim 34, wherein the engaging means comprises a silicone rubber flat ring having a plurality of hooks.

42. The accommodative intraocular lens of claim 34, wherein the engaging means comprises a plurality of ridge bars.

43. An accommodative intraocular lens for implantation in an eye of a living subject having a lens capsule and a lens substance contained in the lens capsule, comprising:
a. a lens structure defining a volume, the volume filled with an optical transparent liquid; and
b. a ring frame engaging the lens structure at an edge with a radius at a plurality of positions and the lens capsule at an equator at a plurality of positions.

44. A method of constructing an accommodative intraocular lens for implantation in an eye of a living subject having a lens capsule and a lens substance contained in the lens capsule, comprising the steps of:
a. forming a lens structure having a geometry and a focal power associated with the geometry, the lens geometry being changeable in response to a force applied to the lens structure;
b. forming a frame; and
c. engaging the frame with the lens structure and the lens capsule of the eye of the living subject such that contraction of the lens capsule pushes the lens structure contracting inwardly and relaxation of the lens capsule pulls the lens structure extending outwardly so as to change the lens geometry of the lens structure to adjust the focal power of the lens structure accordingly.

45. The method of claim 44, wherein the step of forming a lens structure comprises the step of:
a. forming a first film and a second film, each of the first film and the second film having an edge;
b. attaching the edge of the first film to the edge of the second film to form a volume therebetween the first film and the second film; and
c. filling a gel into the volume.

46. The method of claim 45, wherein the first film and the second film are made of an elastic silicone rubber.

47. The method of claim 46, wherein the elastic silicone rubber comprises one of an elastomeric polydimethylsiloxane and a hydrogel.

48. The method of claim 45, wherein the gel comprises a liquid gel.
